SEQUENCE LISTING (1 sequence)

SEQ ID NO: 1 - protein, 960 aa, Aspergillus niger
STTAPSQPQFTIPASADVGAQLIANIDDPQAADAQSVCPGYKASKVQHNSRGFTASLQLAGRPCNVYGTDVESLTLSVEYQDSDRLNIQILPTHVDSTNASWYFLSENLVPRPKASLNASVSQSDLFVSWSNEPSFNFKVIRKATGDALFSTEGTVLVYENQFIEFVTALPEEYNLYGLGEHITQFRLQRNANLTIYPSDDGTPIDQNLYGQHPFYLDTRYYKGDRQNGSYIPVKSSEADASQDYISLSHGVFLRNSHGLEILLRSQKLIWRTLGGGIDLTFYSGPAPADVTRQYLTSTVGLPAMQQYNTLGFHQCRWGYNNWSDLADVVANFEKFEIPLEYIWTDIDYMHGYRNFDNDQHRFSYSEGDEFLSKLHESGRYYVPIVDAALYIPNPENASDAYATYDRGAADDVFLKNPDGSLYIGAVWPGYTVFPDWHHPKAVDFWANELVIWSKKVAFDGVWYDMSEVSSFCVGSCGTGNLTLNPAHPSFLLPGEPGDIIYDYPEAFNITNATEAASASAGASSQAAATATTTSTSVSYLRTTPTPGVRNVEHPPYVINHDQEGHDLSVHAVSPNATHVDGVEEYDVHGLYGHQGLNATYQGLLEVWSHKRRPFIIGRSTFAGSGKWAGHWGGDNYSKWWSMYYSISQALSFSLFGIPMFGADTCGFNGNSDEELCNRWMQLSAFFPFYRNHNELSTIPQEPYRWASVIEATKSAMRIRYAILPYFYTLFDLAHTTGSTVMRALSWEFPNDPTLAAVETQFMVGPAIMVVPVLEPLVNTVKGVFPGVGHGEVWYDWYTQAAVDAKPGVNTTISAPLGHIPVYVRGGNILPMQEPALTTREARQTPWALLAALGSNGTASGQLYLDDGESIYPNATLHVDFTASRSSLRSSAQGRWKERNPLANVTVLGVNKEPSAVTLNGQAVFPGSVTYNSTSQVLFVGGLQNLTKGGAWAENWVLEW

(19) United States
(12) Patent Application Publication
(10) Pub. No.: US 2025/0011694 A1
(43) Pub. Date: Jan. 9, 2025

(54) PRODUCTION OF HIGHLY ATTENUATED BEERS

(71) Applicant: INTERNATIONAL N&H DENMARK APS, Kongens Lyngby (DK)

(72) Inventors: Jacob Flyvholm Cramer, Højbjerg (DK); Jannik Munksgaard, Kongens Lyngby (DK)

(21) Appl. No.: 18/711,238

(22) PCT Filed: Nov. 22, 2022
(86) PCT No.: PCT/US2022/080289

§ 371 Date: May 17, 2024

Related U.S. Application Data
(60) Provisional application No. 63/282,709, filed on Nov. 24, 2021.

(51) Int. Cl. C12C 5/00; C12C 11/00; C12N 9/10; C12N 9/34; C12P 19/18

(52) U.S. Cl. CPC C12C 5/004; C12C 11/00; C12N 9/1051; C12N 9/2428; C12P 19/18; C12Y 204/01024; C12Y 302/01003

(57) ABSTRACT

The present invention relates to a method for increasing attenuation in a fermented beverage. More particularly, the method relates to addition of a transglucosidase to a maltose depleted fermentate to convert non-fermentable dextrins to maltose.

### CROSS REFERENCE TO RELATED APPLICATIONS

This application claims the benefit of U.S. Provisional Patent Application No. 63/282,709, filed Nov. 24, 2021, which is incorporated herein by reference in its entirety.

### SEQUENCE LISTING

The sequence listing filed herewith named “NB42015-WO-PCT_SequenceListing.xml” was created on Nov. 21, 2022 and is 3 KB in size. The sequence listing is incorporated herein by reference in its entirety.

### BACKGROUND OF THE INVENTION

In the production of beer, during the mashing step, fermentable sugars are generated from starch which are subsequently converted into ethanol by yeast. However, not all starch is converted into fermentable sugars. Short glucose oligomers remain after fermentation which cannot be converted into ethanol by brewer's yeast. These oligomers, also called dextrins, are usually 4 sugar units and above (DP4+). As dextrins are not converted into ethanol, these sugars are present in the resulting beer, increasing the caloric content of the beer. Many consumers consider high calories beers to be undesirable.

There remains a need in the art for methods to produce beers having less non-fermentable sugars and a lower caloric content.

### SUMMARY OF THE INVENTION

In accordance with an aspect of the present invention, a method is presented for increasing attenuation in a fermented beverage by adding a transglucosidase to a maltose depleted fermentate.

Optionally, the fermented beverage is a beer.

Optionally, the fermentate has a concentration of maltose of less than 0.01% (w/w).

Optionally, less than 3 U/ml, 1 U/ml or 0.5 U/ml of transglucosidase is added to the fermentate.

Optionally, the transglucosidase is added after more than 50% of the total fermentation time.

Optionally, the beer has an RDF of at least 85, 86, 87, 88, or 89%.

Optionally, the beer has an apparent extract of −0.20% or less by mass.

Optionally, the transglucosidase is added to the fermentate 4 days after initiation of fermentation.

Optionally, a glucoamylase is also added to the fermentate. Optionally, the glucoamylase is added at the start of fermentation.

Optionally, the transglucosidase is a polypeptide having 70% or more sequence identity to SEQ ID NO: 1 or a transglucosidase active fragment thereof.

Optionally, the polypeptide has 75% or more sequence identity to SEQ ID NO:1 or a transglucosidase active fragment thereof.

Optionally, the polypeptide has 80% or more sequence identity to SEQ ID NO:1 or a transglucosidase active fragment thereof.

Optionally, the polypeptide has 85% or more sequence identity to SEQ ID NO:1 or a transglucosidase active fragment thereof.

Optionally, the polypeptide has 90% or more sequence identity to SEQ ID NO:1 or a transglucosidase active fragment thereof.

Optionally, the polypeptide has 95% or more sequence identity to SEQ ID NO:1 or a transglucosidase active fragment thereof.

Optionally, the polypeptide has 99% or more sequence identity to SEQ ID NO: 1 or a transglucosidase active fragment thereof.

Optionally, the polypeptide has 100% sequence identity to SEQ ID NO: 1 or a transglucosidase active fragment thereof.

### BRIEF DESCRIPTION OF SEQ ID NOS

SEQ ID NO: 1 sets forth the mature amino acid sequence of the alpha-glucosidase (transglucosidase) from Aspergillus niger.

### DEFINITIONS and ABBREVIATIONS

Unless defined otherwise, all technical and scientific terms used herein have the same meaning as commonly understood by one of ordinary skill in the art to which this disclosure belongs. Singleton et al., DICTIONARY OF MICROBIOLOGY AND MOLECULAR BIOLOGY, 2^{nd} ed., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

As used herein, the term “glucoamylase (EC 3.2.1.3)” refers to an enzyme that catalyzes the release of D-glucose from the non-reducing ends of starch and related oligo- and polysaccharides.

A “variant” or “variants” refers to either polypeptides or nucleic acids. The term “variant” may be used interchangeably with the term “mutant”. Variants include insertions, substitutions, transversions, truncations, and/or inversions at one or more locations in the amino acid or nucleotide sequence, respectively. The phrases “variant polypeptide”, “polypeptide variant”, “polypeptide”, “variant” and “variant enzyme” mean a polypeptide/protein that has an amino acid sequence that either has or comprises a selected amino acid sequence of or is modified compared to the selected amino acid sequence, such as SEQ ID NO: 1, 2, 3, 4 or 5.

As used herein, a “homologous sequence” and “sequence identity” with regard to a nucleic acid or polypeptide sequence means having about at least 100%, at least 99%, at least 98%, at least 97%, at least 96%, at least 95%, at least 94%, at least 93%, at least 92%, at least 91%, at least 90%, at least 88%, at least 85%, at least 80%, at least 75%, at least 70%, at least 65%, at least 60%, at least 55%, at least 50%, or at least 45% sequence identity to a nucleic acid sequence or polypeptide sequence when optimally aligned for comparison, wherein the function of the candidate nucleic acid sequence or polypeptide sequence is essentially the same as the nucleic acid sequence or polypeptide sequence the candidate homologous sequence is being compared with. In some embodiments, homologous sequences have between at least about 85% and 100% sequence identity, while in other embodiments there is between about 90% and 100% sequence identity, and in other embodiments, there is at least about 95% and 100% sequence identity.

Homology is determined using standard techniques known in the art (see e.g., Smith and Waterman, Adv. Appl. Math. 2:482 (1981); Needleman and Wunsch, J. Mol. Biol. 48:443 (1970); Pearson and Lipman, Proc. Natl. Acad. Sci. USA 85:2444 (1988); programs such as GAP, BESTHT, FASTA, and TFASTA in the Wisconsin Genetics Software Package (Genetics Computer Group, Madison, WI); and Devereux et al., Nucleic Acid Res., 12:387-395 (1984)).

The “percent (%) nucleic acid sequence identity” or “percent (%) amino acid sequence identity” is defined as the percentage of nucleotide residues or amino acid residues in a candidate sequence that is identical with the nucleotide residues or amino acid residues of the starting sequence. The sequence identity can be measured over the entire length of the starting sequence.

Homologous sequences are determined by known methods of sequence alignment. A commonly used alignment method is BLAST described by Altschul et al., (Altschul et al., J. Mol. Biol. 215:403-410 (1990); and Karlin et al, Proc. Natl. Acad. Sci. USA 90:5873-5787 (1993)). A particularly useful BLAST program is the WU-BLAST-2 program (see Altschul et al, Meth. Enzymol. 266:460-480 (1996)). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched. However, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Other methods find use in aligning sequences. One example of a useful algorithm is PILEUP. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle (Feng and Doolittle, J. Mol. Evol. 35:351-360 (1987)). The method is similar to that described by Higgins and Sharp (Higgins and Sharp, CABIOS 5:151-153 (1989)). Useful PILEUP parameters including a default gap weight of 3.00, a default gap length weight of 0.10, and weighted end gaps. The term “optimal alignment” refers to the alignment giving the highest percent identity score.

As used herein, the term “beer” traditionally refers to an alcoholic beverage derived from malt, which is derived from barley, and optionally adjuncts, such as cereal grains, and flavoured with hops. Beer can be made from a variety of grains by essentially the same process. All grain starches are glucose homopolymers in which the glucose residues are linked by either alpha-1,4- or alpha-1,6-bonds, with the former predominating. The process of making fermented malt beverages is commonly referred to as brewing. The principal raw materials used in making these beverages are water, hops and malt. In addition, adjuncts such as common corn grits, refined corn grits, brewer's milled yeast, rice, sorghum, refined corn starch, barley, barley starch, dehusked barley, wheat, wheat starch, torrified cereal, cereal flakes, rye, oats, potato, tapioca, and syrups, such as corn syrup, sugar cane syrup, inverted sugar syrup, barley and/or wheat syrups, and the like may be used as a source of starch. The starch will eventually be converted into dextrins and fermentable sugars. For a number of reasons, the malt, which is produced principally from selected varieties of barley, has the greatest effect on the overall character and quality of the beer. First, the malt is the primary flavouring agent in beer. Second, the malt provides the major portion of the fermentable sugar. Third, the malt provides the proteins, which will contribute to the body and foam character of the beer. Fourth, the malt provides the necessary enzymatic activity during mashing.

As used herein, the term “Hops” refers to it use in contributing significantly to beer quality, including flavoring. In particular, hops (or hops constituents) add desirable bittering substances to the beer. In addition, the hops act as protein precipitants, establish preservative agents and aid in foam formation and stabilization.

As used herein, the “process for making beer” is one that is well known in the art, but briefly, it involves five steps: (a) mashing and/or adjunct cooking (b) wort separation and extraction (c) boiling and hopping of wort (d) cooling, fermentation and storage, and (e) maturation, processing and packaging. In the first step, milled or crushed malt is mixed with water and held for a period of time under controlled temperatures to permit the enzymes present in the malt to convert the starch present in the malt into fermentable sugars. In the second step, the mash is transferred to a “lauter tun” or mash filter where the liquid is separated from the grain residue. This sweet liquid is called “wort” and the left over grain residue is called “spent grain”.

The mash is typically subjected to an extraction, which involves adding water to the mash in order to recover the residual soluble extract from the spent grain. In the third step, the wort is boiled vigorously. This sterilizes the wort and helps to develop the colour, flavour and odour. Hops are added at some point during the boiling. In the fourth step, the wort is cooled and transferred to a fermentor, which either contains the yeast or to which yeast is added. After addition of yeast, the liquid is referred to as a fermentate. The yeast converts the sugars by fermentation into alcohol and carbon dioxide gas; at the end of fermentation the fermentor is chilled or the fermentor may be chilled to stop fermentation. The yeast flocculates and is removed. In the last step, the beer is cooled and stored for a period of time, during which the beer clarifies and its flavor develops, and any material that might impair the appearance, flavor and shelf life of the beer settles out. Prior to packaging, the beer is carbonated and, optionally, filtered and pasteurized. After fermentation, a beverage is obtained which usually contains from about 2% to about 10% alcohol by weight. The non-fermentable carbohydrates are not converted during fermentation and form the majority of the dissolved solids in the final beer. This residue remains because of the inability of malt amylases to hydrolyze the alpha-1,6-linkages of the starch. The non-fermentable carbohydrates contribute about 50 calories per 12 ounces of beer.

The term “fermentation” means, in the context of brewing, the transformation of sugars in the wort, by enzymes in the brewing yeast, into ethanol and carbon dioxide with the formation of other fermentation by-products.

As used herein, a “fermentate” is the liquid solution undergoing a fermentation process leading to chemical change of the food, beer or beverage by the action of yeast or bacteria, which produce carbon dioxide and turns carbohydrates in it into alcohol.

As used herein, a “transglucosidase” is synonymous with the term α-glucosidase having predominant transglucosylating activity and the systematic name α-D-glucoside glucohydrolase, having the Enzyme Commission designation EC 3.2.1.20.

As used herein, an “alpha-glucosidase” hydrolyzes or transglucosylate terminal non-reducing (1→4)-linked alpha-glucose residues to produce glucose or IMOs. The exo-acting enzyme is having the systematic name α-D-(1→4)-glucan glucanohydrolase) and the Enzyme Commission designation EC 3.2.1.1.

As used herein, “isomalto-oligosaccharides (IMO)” generally refer to oligosaccharides of glucose that include α-D-1,6 bonds. Exemplary isomalto-oligosaccharides, and their condensed IUPAC name (Id.), include but are not limited to, isomaltose (Glc(α-1,6)Glc), isomaltotriose (Glc(α-1,6) Glc(α-1,6)Glc), and isomaltotetraose (Glc(α-1,6)Glc(α-1,6)Glc(α-1,6)Glc). Branched oligosaccharides having both α-D-1,4 and α-D-1,6 bonds, for example panose (Glc(α-1,6)Glc(α-1,4)Glc) are often considered IMO as well.

As used herein the term “malt” is understood as any malted cereal grain, such as barley.

As used herein, the term “wort” refers to the unfermented liquor run-off following extracting the grist during mashing.

As used herein, the term “spent grains” refers to the drained solids remaining when the grist has been extracted and the wort separated from the mash.

As used herein, the term “beer” refers to fermented wort, e.g. an alcoholic beverage brewed from barley malt, optionally adjunct and hops.

As used herein, the term “extract recovery” in the wort is defined as the sum of soluble substances extracted from the grist (malt and adjuncts) expressed in percentage based on dry matter.

As used herein, the term “pasteurisation” means the killing of micro-organisms in aqueous solution by heating. Implementation of pasteurisation in the brewing process is typically through the use of a flash pasteuriser or tunnel pasteuriser. As used herein, the term “pasteurisation units or PU” refers to a quantitative measure of pasteurisation. One pasteurisation unit (1 PU) for beer is defined as a heat retention of one minute at 60 degrees Celsius. One calculates that:

$$PU = t \times 1.393 ⋀ ( T - 60 ) ,$$

where:
t=time, in minutes, at the pasteurisation temperature in the pasteuriser
T=temperature, in degrees Celsius, in the pasteuriser
[\hat{ }(T−60) represents the exponent of (T−60)]
   Different minimum PU may be used depending on beer type, raw materials and microbial contamination, brewer and perceived effect on beer flavor. Typically, for beer pasteurisation, 14-15 PU are required. Depending on the pasteurising equipment, pasteurisation temperatures are typically in the range of 64-72 degrees Celsius with a pasteurisation time calculated accordingly. Further information may be found in “Technology Brewing and Malting” by Wolfgang Kunze of the Research and Teaching Institute of Brewing, Berlin (VLB), 3rd completely updated edition, 2004, ISBN 3-921690-49-8.

As used herein, the term “DP1” (degree of polymerization 1) means glucose or fructose. “DP2” denotes maltose and/or isomaltose. “DP3” means maltotriose, panose and isopanose. “DP4/4+” means dextrin or maltooligosaccharides of a polymerization degree of 4 or higher which are unfermentable.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Before the exemplary embodiments are described in more detail, it is to be understood that this disclosure is not limited to particular embodiments described, as such may, of course, vary. Although any methods and materials similar or equivalent to those described herein can be used in the practice or testing of the present disclosure, exemplary methods, and materials are now described.

As used herein and in the appended claims, the singular forms “a”, “an”, and “the” include plural referents unless the context clearly dictates otherwise. Thus, for example, reference to “a gene” includes a plurality of such candidate agents and reference to “the cell” includes reference to one or more cells and equivalents thereof known to those skilled in the art, and so forth.

The publications discussed herein are provided solely for their disclosure prior to the filing date of the present application. Nothing herein is to be construed as an admission that the present disclosure is not entitled to antedate such publication by virtue of prior invention.

### DETAILED DESCRIPTION OF THE INVENTION

In accordance with an aspect of the present invention, it has been discovered that a transglucosidase may be employed in brewing to produce highly attenuated beer. Without being bound by theory, it has been discovered in accordance with the instant invention, that a transglucosidase may be added to a fermentate to convert non-fermentable dextrins to maltose which is converted into ethanol. However, the transglucosidase may only be added to the fermentate when maltose carried over from the wort is sufficiently depleted. When the maltose is sufficiently depleted, the transglucosidase will convert un-fermentable dextrin to maltose. However, if the transglucosidase is added to the fermentate prior to sufficient maltose depletion (or even to the wort as taught by the prior art), the transglucosidase will perform the reverse reaction converting maltose to un-fermentable dextrins.

In recent years, changes in the health awareness and preferences of consumers have resulted in increased consumer demand for beer-tasting beverages having low sugar content. In the case of beer or beverages obtained by fermenting of various malt based raw materials, by increasing the amount of fermentable sugar in the fermentation liquid, the final sugar content in the beer or beverage that represents the final product can be reduced.

A transglucosidase (also known as α-glucosidase) may perform transglucosylation in the presence of maltose as substrate, e.g. during mashing or in the final wort. The transglucosidase produces IsoMalto-Oligosaccharides (IMOs) from maltose. These IMOs are generally known to be non-fermentable. Maltose is the donor molecule in the transglycolysation reaction, which hydrolyzes maltose, releasing one free glucose molecule and transferring the other glucose molecule to an acceptor. The acceptor can be another maltose molecule, resulting in a trisaccharide. The most abundant trisaccharide formed is panose. The glucose can also be transferred to a higher sugar, resulting in longer chain isomalto-oligosaccharide, transferred to glucose, resulting in isomaltose formation, or transferred to water, releasing it as another free glucose molecule. The rate at which different oligosaccharides are formed depends on the concentration of the different acceptors.

Most importantly, under conditions where maltose is absent the transglucosidase may hydrolyse these non-fermentable IMO sugars such as: pannose and isomaltose. Thus, these sugars may be converted from non-fermentable to fermentable sugar but only if transglucosidase is applied in absence of maltose that will lead to increased IMOs.

Transglucosidase produces glucose via a hydrolysis reaction, but when the substrate concentration is high, catalyzes a transglucosylation reaction. For example, in a malt beverage using transglucosidase in mashing prior to the heat treatment produces high concentrations of isomaltooligosaccharides such as isomaltose and panose, which are non-fermentable sugars.

Similarly, it is clear that addition of the Transglucosidase to the final wort before fermentation day 4, results in very high generation of the isomaltooligosaccharides: Isomaltose, Nigerose, Gentiobiose and Cellobiose. This is clearly unwanted for brewing low carb or light beers.

An aspect of the present invention concerns the efficient addition of very low dosages (below 3 U/mL) of transglucosidase after at least 4 days of fermentation to convert non-fermentable sugars in the absence of maltose and without production of isomaltooligosaccharides.

In accordance with an aspect of the present invention, a method is presented for increasing attenuation in a fermented beverage by adding a transglucosidase to a maltose depleted fermentate.

Preferably, the fermented beverage is a beer.

Preferably, the fermentate has a concentration of maltose of less than 0.01% (w/w).

Preferably, less than 3 U/ml, 1 U/ml or 0.5 U/ml of transglucosidase is added to the fermentate.

Preferably, the transglucosidase is added after more than 50% of the total fermentation time.

Preferably, the beer has an RDF of at least 85, 86, 87, 88 or 89%.

Preferably, the beer has an apparent extract of −0.20% or less by mass.

Preferably, the transglucosidase is added to the fermentate 4 days after initiation of fermentation.

Preferably, a glucoamylase is also added to the fermentate. Preferably, the glucoamylase is added at the start of fermentation.

Preferably, the transglucosidase is a polypeptide having 70% or more sequence identity to SEQ ID NO: 1 or a transglucosidase active fragment thereof.

Preferably, the polypeptide has 75% or more sequence identity to SEQ ID NO:1 or a transglucosidase active fragment thereof.

Preferably, the polypeptide has 80% or more sequence identity to SEQ ID NO:1 or a transglucosidase active fragment thereof.

Preferably, the polypeptide has 85% or more sequence identity to SEQ ID NO:1 or a transglucosidase active fragment thereof.

Preferably, the polypeptide has 90% or more sequence identity to SEQ ID NO:1 or a transglucosidase active fragment thereof.

Preferably, the polypeptide has 95% or more sequence identity to SEQ ID NO:1 or a transglucosidase active fragment thereof.

Preferably, the polypeptide has 99% or more sequence identity to SEQ ID NO:1 or a transglucosidase active fragment thereof.

Preferably, the polypeptide has 100% sequence identity to SEQ ID NO: 1 or a transglucosidase active fragment thereof.

The present disclosure is described in further detail in the following examples, which are not in any way intended to limit the scope of the disclosure as claimed. The attached figures are meant to be considered as integral parts of the specification and description of the disclosure. The following examples are offered to illustrate, but not to limit the claimed disclosure.

### Example 1—DP Sugar HPLC Analysis of Malt Based Wort Added Alpha-Glucosidase

All standards: Glucose, Maltose, Maltotriose and Maltotetraose were prepared in double distilled water (ddH20) and filtered through 0.45 μm syringe filters. A set of each standard was prepared ranging in concentration from 10 to 100,000 ppm. All wort samples containing active enzymes were inactivated by heating the sample to 95° C. for 10 min. Subsequently wort samples were prepared in 96 well MTP plates (Corning, NY, USA) and diluted minimum 4 times in ddH20 and filtered through 0.20 μm 96 well plate filters before analysis (Corning filter plate, PVDF hydrophile membrane, NY, USA). All samples were analyzed in duplicates. Quantification of sugars: DP1, DP2, DP3, DP4 and DP5+ were performed by HPLC. Analysis of samples was carried out on a Dionex Ultimate 3000 HPLC system (Thermo Fisher Scientific) equipped with a DGP-3600SD Dual-Gradient analytical pump, WPS-3000TSL thermostated autosampler, TCC-3000SD thermostated column oven, and a RI-101 refractive index detector (Shodex, JM Science). Chromeleon datasystem software (Version 6.80, DU10A Build 2826, 171948) was used for data acquisition and analysis.

### Chromatographic Conditions

The samples were analyzed using a RSO oligosaccharide column, Ag+4% crosslinked (Phenomenex, The Netherlands) equipped with an analytical guard column (Carbo-Ag+ neutral, AJ0-4491, Phenomenex, The Netherlands) operated at 70° C. The column was eluted with double distilled water (filtered through a regenerated cellulose membrane of 0.45 μm and purged with helium gas) at a flow rate of 0.3 ml/min. Isocratic flow of 0.3 ml/min was maintained throughout analysis with a total run time of 45 min and injection volume was set to 10 μL. Samples were held at 20. C in the thermostated autosampler compartment. The eluent was monitored by means of a refractive index detector (RI-101, Shodex, JM Science) and quantification was made by the peak area relative to the peak area of the given standard (DP1: glucose; DP2: maltose; DP3: maltotriose and peaks with a degree of four or higher maltotetraose was used as standard).

A 0.5 M MES pH 5.5 stock was prepared as follows, 4.881 g MES powder (Sigma Aldrich, M8250) was dissolved into 40 ml MilliQ. pH was adjusted using 10% w/w NaOH and volume filled to 50.0 ml. Diluted Muntons Malt extract (Munton's Light Malt Extract, Batch XB 35189) was prepared by dissolving 25 g Muntons malt extract in 25 g 0.5 M MES buffer pH 5.5. A 100× stock dilution of alpha-glucosidase was prepared by diluting 0.2 g enzyme (FoodPro® TGO, Dupont Nutrition Bioscience, Denmark having an activity of 2000 U/g) in 20 mL MES pH5.5. The wort sample was prepared by mixing 600 μL diluted Muntons extract, 100 μL 0.5M MES pH5.5, 262.5 μL MilliQ water and 37.5 μL diluted alpha-glucosidase (or water as no enzyme control. Samples were incubated at 60° C. in a thermomixer at 750 rpm to evaluate an accelerated DP sugar conversion. Samples were taken at 30 min intervals and eventual enzyme activity was stopped by incubation at 95 C for 15 min in a thermomixer at 750 rpm and followingly frozen before HPLC sugar DP analysis.

The relative distribution of sugars from HPLC analysis of wort with alpha-glucosidase is shown in Table 1 below. It is clearly seen that maltose (DP2) is the major sugar in the malt-based wort. The accelerated DP sugar conversion in presence alpha-glucosidase in the wort (having maltose) clearly demonstrate the generation of the non-fermentable IMOs (DP3+DP4+) throughout the reaction time (4 hrs). Generation of IMOs in the wort or during fermentation is not preferred to obtain high attenuation. Thus alpha-glucosidase should not be added in presence of maltose.

TABLE 1
Relative distribution of sugars from HPLC
analysis of wort with alpha-glucosidase.
% Relative distribution of sugars

| Sample | | | | | Total | IMOs |
|---|---|---|---|---|---|---|
| timepoint (Hours) | DP4+ | DP3 | DP2 | DP1 | Sugar | (DP3+) |
| 0 | 20 | 14 | 55 | 11 | 100 | 35 |
| 0.5 | 19 | 22 | 46 | 13 | 100 | 41 |
| 1 | 20 | 25 | 39 | 15 | 100 | 46 |
| 1.5 | 20 | 27 | 36 | 16 | 100 | 48 |
| 2 | 21 | 28 | 33 | 18 | 100 | 49 |
| 2.5 | 21 | 27 | 33 | 19 | 100 | 48 |
| 3 | 21 | 28 | 32 | 19 | 100 | 49 |
| 4 | 22 | 27 | 31 | 21 | 100 | 49 |

### Example 2—DP Sugar HPLC Analysis of Fermentate Samples

All standards: Glucose, Maltose, Maltotriose and Maltotetraose were prepared in double distilled water (ddH20) and filtered through 0.45 μm syringe filters. A set of each standard was prepared ranging in concentration from 10 to 100,000 ppm. All wort samples containing active enzymes were inactivated by heating the sample to 95° C. for 10 min. Subsequently wort samples were prepared in 96 well MTP plates (Corning, NY, USA) and diluted minimum 4 times in ddH20 and filtered through 0.20 μm 96 well plate filters before analysis (Corning filter plate, PVDF hydrophile membrane, NY, USA). All samples were analyzed in duplicates.

Quantification of sugars: DP1, DP2, DP3 and DP4+ were performed by HPLC. Analysis of samples was carried out on a Dionex Ultimate 3000 HPLC system (Thermo Fisher Scientific) equipped with a WPS-3000TSL thermostated autosampler, TCC-3000SD thermostated column oven, and a RI-101 refractive index detector (Shodex, JM Science). Chromeleon datasystem software (Version 6.80, DU10A Build 2826, 171948) was used for data acquisition and analysis.

### Chromatographic Conditions

The samples were analyzed using a RSO oligosaccharide column, Ag⁺ 4% crosslinked (Phenomenex, The Netherlands) equipped with an analytical guard column (Carbo-Ag⁺ neutral, AJ0-4491, Phenomenex, The Netherlands) operated at 70° C. The column was eluted with double distilled water (filtered through a regenerated cellulose membrane of 0.45 μm and purged with helium gas) at a flow rate of 0.3 ml/min. Isocratic flow of 0.3 ml/min was maintained throughout analysis with a total run time of 45 min and injection volume was set to 10 μL. Samples were held at 20° C. in the thermostated autosampler compartment. The eluent was monitored by means of a refractive index detector (RI-101, Shodex, JM Science) and quantification was made by the peak area relative to the peak area of the given standard (DP1: glucose; DP2: maltose; DP3: maltotriose and peaks with a degree of four or higher maltotetraose was used as standard).

### Example 3—Reduction in Beer Carbohydrate Content by Conversion of Non-Fermentable Sugars During Beer Fermentation by Alpha-Glucosidase

The objective of this analysis was to test the addition of a transglucosidase during fermentation specifically at low maltose concentration to enable hydrolytic conversion of wort non-fermentable sugars into fermentable sugars to proceed in parallel with ethanol formation by yeast.

Wort was prepared from mashing operation with 50% Pilsner malt (Pilsner malt; Fuglsang Denmark, Batch Number: Oct. 12, 2019) and 50% Corn grits (Nordgetreide GmBH Lübec, Germany, Batch: Feb. 5, 2016.), using a water to grist ratio of 3:1. Pilsner malt was milled at a Buhler Miag mill (0.5 mm setting).

The corn adjunct was liquefied in the follow way: Corn grits (35.0 g), Malt (milled pilsner malt, 5.5 g) and tap water (105 g) was mixed in mashing bath (Lockner, LG-electronics) cups and pH adjusted to pH 5.5 with 2.5M sulphuric acid. AMYLEX® 5T (Dupont Nutrition Bioscience, Denmark) was added at a dosage of 0.25 kg/t grist to facilitate liquefaction.

The adjunct was mashed with the program; heated to 60° C. and kept for 1 minute for mashing in; heated to 85° C. for 13 minutes by increasing temperature with 2° C./minute; kept at 85° C. for 30 minutes and mashing off. Hereafter the adjunct was cooled to 64° C. and combined with the main mash. LAMINEX® MaxFlow 4G (Dupont Nutrition Bioscience, Denmark) was added at a dosage of 0.10 kg/t malt to facilitate filtration. DIAZYME® 87 (Dupont Nutrition Bioscience, Denmark) was added at a dosage of 0.0, 1.5 or 3.0 kg/t malt to facilitate saccharification.

In the main mash, malt (milled pilsner malt, 35 g) and tap water (105 g) was mixed in a mashing bath (Lochner, LG-electronics) cup and pH adjusted to pH 5.5 with 2.5M sulphuric acid. The main mash was heated to heated to 45° C. and kept for 1 minute for mashing in before heated to 63° C. and main mash was combined with adjunct (liquefied corn, malt and water); kept at 63° C. for 120 minutes and heated to 72° C. for 9 minutes by increase temperature with 1° C./minute; kept at 72° C. for 15 minutes and heated to 78° C. for 6 minutes by increase temperature with 1° C./minute. The mash was finally held at 78° C. for 10 min and mashing-off. Iodine negative was tested when temperature had reached 72° C. The time in minutes that was required to get iodine negative was noted.

At the end of mashing, the mashes were made up to 350 g and filtered. Filtrate volumes were measured after 30 minutes. The pH was adjusted to pH 5.2 with 2.5 M sulphuric acid and one pellet of bitter hops from Hopfenveredlung, St. Johann: Alpha content of 16.0% (EBC 7.7 0 specific HPLC analysis, Jan. 10, 2013), was added to each flask (210 g). The wort samples were boiled for 60 minutes in a boiling bath and wort were cooled down to 17° C. and filtered. 100 g of each wort was weighted out into a 500 ml conical flask for fermentation adding 0.5% W34/70 (Weihenstephan) freshly produced yeast (0.50 g) to the wort having 17° C. The remaining of the filtered wort was used for analysis. The wort samples were fermented at 18° C. and 150 rpm after yeast addition. To ensure high degree of attenuation and low content of carbohydrates in the final beer two different glucoamylase were added wort in the start of the fermentation: DIAZYME® 87 (Dupont Nutrition Bioscience, Denmark) was added at a dosage of 0.5 g/hl or DIAZYME® TGA (Dupont Nutrition Bioscience, Denmark) was added at a dosage of 1.0 g/hl respectively. The addition of glucoamylase in that start of fermentation was combined with the addition of a transglucosidase FoodPro® TGO (Dupont Nutrition Bioscience, Denmark) with an activity of 2000 U/g in a low dose of 0.02 U/mL (1.0 g product/hL) specifically added after 4 days. The combinations of enzymes are shown in table 2 below. All fermentation lasted 10 days. Analysis was performed when fermentation had finished.

TABLE 2
Addition of enzymes during adjunct, mashing and fermentation.

| | | Mashing | | Start Fermentation | | 4 days |
|---|---|---|---|---|---|---|
| | Adjunkt | LAMINEX ® | DIAZYME ® | DIAZYME ® | DIAZYME ® | Fermentation |
| | Amylex 5T | MaxFlow 4G | 87 | 87 | TGA | FPro TGO |
| Sample no. | (kg/t grist) | (kg/t grist) | (kg/t) | (g/hl) | (U/mL) | (g/hl) |
| 1 | 0.25 | 0.10 | 3.0 | | | |
| 2 | 0.25 | 0.10 | 3.0 | | | 1.0 |
| 3 | 0.25 | 0.10 | 1.5 | 0.5 | | |
| 4 | 0.25 | 0.10 | 1.5 | 0.5 | | 1.0 |
| 5 | 0.25 | 0.10 | | | 0.02 | |
| 6 | 0.25 | 0.10 | | | 0.02 | 1.0 |

Wort analysis: Original Extract (OE), Extract in the wort samples after mashing was measured using Anton Paar (Lovis) following Dupont Standard Instruction Brewing, 23.8580-B28 and Fermentable sugars (% total+g/100 ml) by HPLC were DP1, DP2, DP3 and DP4+ was determined after mashing following example 2 above. The relative sugar distribution in wort is shown below in table 3.

TABLE 3
Relative distribution of sugars from HPLC analysis of wort.

| | | Mashing | | | | | |
|---|---|---|---|---|---|---|---|
| | Adjunct | LAMINEX ® | | | | | |
| | Amylex | MaxFlow | DIAZYME ® | Relative distribution of sugars in % | | | |
| Mashing | 5T | 4G | 87 | DP1 | DP2 | DP3 | DP4+ |
| position | (kg/t) | (kg/t) | (kg/t) | % | % | % | % |
| 1 | 0.25 | 0.10 | 3.0 | 88.1 | 4.2 | 1.8 | 5.9 |
| 2 | 0.25 | 0.10 | 3.0 | 88.3 | 4.2 | 1.7 | 5.8 |
| 3 | 0.25 | 0.10 | 1.5 | 67.8 | 18.6 | 2.7 | 10.9 |
| 4 | 0.25 | 0.10 | 1.5 | 67.2 | 19.1 | 2.6 | 11.2 |
| 5 | 0.25 | 0.10 | 0.0 | 9.4 | 50.1 | 18.2 | 22.2 |
| 6 | 0.25 | 0.10 | 0.0 | 9.5 | 50.2 | 18.5 | 21.8 |

Beer analysis: RDF was measured using an Anton Paar (DMA 5000) following Standard Instruction Brewing, 23.8580-B28 and alcohol by Dupont Standard Instruction Brewing, 23.8580-B28. Real degree of fermentation (RDF) value may be calculated according to the equation below:

$$R D F ( \% ) = ( 1 - \frac{R E}{{}^{o} {P}_{initial}} ) \times 100$$

Where: RE=real extract=(0.1808×°Pᵢₙᵢₜᵢₐₗ)+(0.8192×°P_{final}), °Pᵢₙᵢₜᵢₐₗ is the specific gravity of the standardised worts before fermentation and °P_{final} is the specific gravity of the fermented worts expressed in degree Plato.

In the present context, Real degree of fermentation (RDF) was determined from the specific gravity and alcohol concentration.
Specific gravity and alcohol concentration was determined on the fermented samples using a Beer Alcolyzer Plus and a DMA 5000 Density meter (both from Anton Paar, Gratz, Austria). Based on these measurements, the real degree of fermentation (RDF) value was calculated according to the equation below:

$$R D F ( \% ) = \frac{O E - E ( r )}{O E} \times 100$$

Where: E(r) is the real extract in degree Plato (°P) and OE is the original extract in °P. Original Extract (OE) Extract in the beer samples after mashing was measured using an Anton Paar (DMA 5000) following Dupont Standard Instruction Brewing, 23.8580-B28. Analysis was performed when fermentation had finished and yeast was separated, the results are show in table 4. Surprisingly it can be seen that the addition of the transglucosidase at the fourth day of fermentation in all combinations increased the % RDF further by conversion of non-fermentable sugars. Thus, it can be observed by sample 1 and 2, that the addition of the transglucosidase increased % RDF from 80.69% to 85.49% and similarly sample 3 and 4, that the addition of the transglucosidase increased % RDF from 86.56% to 87.43% and lastly sample 5 and 6, that the addition of the transglucosidase increased % RDF from 87.45% to 88.59%. In all cases were the relative high attenuation increased further also observed by the % (v/v) alcohol increase by the addition of the transglucosidase.

TABLE 4
Density (g/cm3), Specific gravity (20/20), Extract (°P), RDF (%) and alcohol
content (% v/v) of fermentation samples with additions of glucoamylase (DIAZYME ® 87
or DIAZYME ® TGA) and/or transglucosidase (FoodPro ® TGO).

| Fermentation | | | | | | | | |
|---|---|---|---|---|---|---|---|---|
| | DIAZYME ® | DIAZYME ® | FPro | | | Extract | | |
| Sample | 87 | TGA | TGO | Densitet | SG | (°P) | RDF | Alcohol |
| no. | (g/hl) | (g/hl) | (U/mL) | (g/cm3) | (20/20) | OE | (%) | % V/V |
| 1 | | | | 0.99848 | 1.00028 | 17.17 | 80.69 | 9.31 |
| 2 | | | 0.02 | 0.99460 | 0.99639 | 17.27 | 85.49 | 9.89 |
| 3 | 0.5 | | | 0.99375 | 0.99554 | 17.23 | 86.56 | 9.97 |
| 4 | 0.5 | | 0.02 | 0.99303 | 0.99482 | 17.29 | 87.43 | 10.10 |
| 5 | | 1.0 | | 0.99297 | 0.99476 | 17.44 | 87.45 | 10.21 |
| 6 | | 1.0 | 0.02 | 0.99211 | 0.99389 | 17.27 | 88.59 | 10.22 |

### Example 4—Non-Fermentable Sugar Analysis in Fermentate Sample by HPLC-PAD

The objective of this analysis was to quantify minor fermentable and non-fermentable sugars upon addition of a transglucosidase and glucoamylase during fermentation. Samples were prepared as described in example 3.

All standards: glucose, isomaltose, isomaltose, maltose, panose, maltotriose, other DP2 and larger saccharides DP4+ were prepared in double distilled water (ddH20) and filtered through 0.45 μm syringe filters. A set of each standard was prepared ranging in concentration from 0.5 to 20 mg/L. All wort samples containing active enzymes were inactivated by heating the sample to 95° C. for 10 min. Subsequently samples were diluted and filtered (0.45 μm Minispike filter) before analysis. All samples were analyzed in duplicates. Quantification of sugars was performed by HPLC-PAD and analysis was carried out on a Diones IC system with PAD detector (Thermo Fisher Scientific) with Chromeleon datasystem software (Version 7.2) for data acquisition and analysis.

### Chromatographic Conditions

The samples were analyzed using Carbo PA100 2 mm column with guard column (Thermo Fisher Scientific) operated at 0.25 mL/min and the elution gradient program in table 5, shown below.

TABLE 5
Elution gradient of Carbo PA100, carbohydrate analysis

| | | | 1 M Na-acetat |
|---|---|---|---|
| Time (min) | Water (%) | 1 M NaOH (%) | (%) |
| 0.000 | 89.0 | 10.0 | 1.0 |
| 12.000 | 85.0 | 10.0 | 5.0 |
| 55.000 | 72.0 | 10.0 | 18.0 |
| 60.000 | 65.0 | 10.0 | 25.0 |
| 65.000 | 89.0 | 10.0 | 1.0 |
| 75.000 | 89.0 | 10.0 | 1.0 |

The injection volume was set to 20 μL flow of 0.25 ml/min was maintained throughout analysis (total run time 75 min). The eluent was monitored by means of a PAD detector (Thermo Fisher Scientific) and quantification was made by the peak area relative to the peak area of the given standard. The concentration of fermentable and non-fermentable sugars and saccharides in the resulting beer samples after 10 day fermentation with or without addition of the transglucosidase are shown in table 6. It can surprisingly, be observed that all non-fermentable saccharides, e.g. isomaltose, isomaltotriose, pannose, maltotriose and other DP4+ saccharides in all samples with addition of transglucosidase added after the fourth day of fermentation (sample 2, 4 and 6) are decreased as compared to the comparable samples without addition of transglucosidase respectively (sample 1, 3 and 5). The same is also observed for other DP2 components. The total sum of all saccharides (fermentable and non-fermentable) are notable decreased by the addition of transglucosidase added after the fourth day of fermentation; e.g. sample 1 without transglucosidase (1.053%) vs sample 2 with transglucosidase (0.253%), sample 3 without transglucosidase (0.235%) vs sample 4 with transglucosidase (0.144%) and 5 without transglucosidase (0.265%) vs sample 6 with transglucosidase (0.206%). This is in agreement with increased % RDF and attenuation of the beer produced with transglucosidase, resulting in higher fermentability and lower resulting saccharides and sugar in the final beer.

TABLE 6
Concentration of glucose, isomaltose, other DP2, isomaltose, maltose, panose, maltotriose and larger saccharides DP4+ (%
w/v) in fermentation samples with additions of glucoamylase (DIAZYME ® 87 or DIAZYME ® TGA) and/or transglucosidase (FoodPro ® TGO).

| | Enzyme added | | Enzyme added | | | | | | | | |
|---|---|---|---|---|---|---|---|---|---|---|---|
| | Fermentation | | Fermentation | | | | | | | | |
| | start | | day 4 | | | | | | | | |
| | DIAZYME ® | DIAZYME ® | FPro | %(w/v) | | | | | | | |
| Sample | 87 | TGA | TGO | | | Other | | | | | |
| no. | (g/hl) | (g/hl) | (U/mL) | Glucose | Isomaltose | DP2 | Isomaltotriose | Maltose | Panose | Maltotriose | DP4+ |
| 1 | | | | 0.012 | 0.090 | 0.060 | <0.01 | 0.431 | 0.14 | 0.028 | 0.25 |
| 2 | | | 0.02 | 0.040 | <0.01 | <0.01 | ND | ND | ND | 0.010 | 0.18 |
| 3 | 0.5 | | | 0.033 | 0.055 | 0.030 | ND | ND | 0.019 | 0.010 | 0.088 |
| 4 | 0.5 | | 0.02 | 0.040 | <0.01 | <0.01 | ND | ND | ND | <0.01 | 0.074 |
| 5 | | 1 | | 0.073 | 0.037 | 0.013 | ND | ND | 0.023 | <0.01 | 0.11 |
| 6 | | 1 | 0.02 | 0.057 | ND | <0.01 | ND | ND | 0.001 | 0.01 | 0.12 |

Although the foregoing invention has been described in some detail by way of illustration and example, for purposes of clarity of understanding, certain changes and modifications can be practiced within the scope of the appended claims. In addition, each reference provided herein is incorporated by reference in its entirety for all purposes to the same extent as if each reference was individually incorporated by reference. To the extent the content of any citation, including website or accession number may change with time, the version in effect at the filing date of this application is meant. Unless otherwise apparent from the context any step, element, aspect, feature of embodiment can be used in combination with any other.

### Example 5—Reduction in Beer Carbohydrate Content by Conversion of Non-Fermentable Sugars During Beer Fermentation by Various Alpha-Glucosidases

The objective of this analysis was to test the addition of a transglucosidase during fermentation specifically at low maltose concentration to enable hydrolytic conversion of wort non-fermentable sugars into fermentable sugars to proceed in parallel with ethanol formation by yeast.

Wort was produced as described in example 3, however in the absence of saccharifying enzymes added in mashing. To ensure high degree of attenuation and low content of carbohydrates in the final beer a glucoamylase was added wort in the start of the fermentation: DIAZYME® 87 (Dupont Nutrition Bioscience, Denmark) was added at a dosage of 3.0 g/hl. The addition of glucoamylase in that start of fermentation was combined with the addition two various transglucosidases: FoodPro® TGO (Dupont Nutrition Bioscience, Denmark) with an activity of 2000 U/g in dosages of 0.01, 0.02 or 0.03 U/mL or Transglucosidase L, manufactured by Amano Pharmaceutical Co., Ltd in dosages of dosages of 0.5, 1.0 or 1.5 g/hL respectively. The transglucosidases were added specifically added after 4 days to ensure low maltose concentration. The combinations of enzymes are shown in table 7 below. All fermentation lasted 10 days. Analysis was performed when fermentation had finished, as described in example 3.

It can be seen, that the addition of the transglucosidase at the fourth day of fermentation in all combinations increased the % RDF further by conversion of non-fermentable sugars. Thus, it can be observed by sample 1 and 2 to 7, that the addition of the transglucosidase increased % RDF from 84.39% to more than 85.4% irrespective of the transglucosidase used. A dose-response effect was seen with addition of FoodPro TGO increasing % RDF from 84.39% to 85.76%. In all cases were the relative high attenuation increased further also observed by the % (v/v) alcohol increase by the addition of the transglucosidase. Thus transglucosidase may be added late in fermentation to increase the amount of fermentable sugars to increase % RDF and alcohol concentration in the final beer.

TABLE 7
Addition of enzymes during mashing and fermentation.

| | | Fermentation | | |
|---|---|---|---|---|
| | Mashing | | Transglucosidase | FPro TGO (4 |
| | LAMINEX ® | DIAZYME ® | L (4 days of | days of |
| | MaxFlow 4G | 87 (Start) | fermentation) | fermentation) |
| Sample no. | (kg/t) | (g/hl) | (g/hl) | (U/mL) |
| 1 | 0.10 | 3.00 | | |
| 2 | 0.10 | 3.00 | 0.5 | |
| 3 | 0.10 | 3.00 | 1 | |
| 4 | 0.10 | 3.00 | 1.5 | |
| 5 | 0.10 | 3.00 | | 0.01 |
| 6 | 0.10 | 3.00 | | 0.02 |
| 7 | 0.10 | 3.00 | | 0.3 |

TABLE 8
Density (g/cm3), Specific gravity (20/20), Extract (°P), RDF (%) and alcohol
content (% v/v) of fermentation samples with additions of glucoamylase (DIAZYME ® 87)
and/or transglucosidase (FoodPro ® TGO or Transglucosidase L).

| | Mashing | | | | | | | | |
|---|---|---|---|---|---|---|---|---|---|
| | LAMINEX ® | Fermentation | | | | | | | |
| | MaxFlow | DIAZYME ® | Transglucosidase | FPro | | | Extract | | |
| Sample | 4G | 87 | L | TGO | Densitet | SG | (°P) | RDF | Alcohol |
| no. | (kg/t) | (g/hl) | (g/hl) | (U/mL) | (g/cm3) | (20/20) | OE | (%) | % V/V |
| 1 | 0.10 | 3.00 | | | 0.99 | 1.00 | 16.12 | 84.39 | 8.91 |
| 2 | 0.10 | 3.00 | 0.5 | | 0.99 | 1.00 | 15.91 | 85.40 | 9.04 |
| 3 | 0.10 | 3.00 | 1 | | 0.99 | 1.00 | 16.21 | 85.71 | 9.25 |
| 4 | 0.10 | 3.00 | 1.5 | | 0.99 | 1.00 | 15.73 | 85.53 | 8.93 |
| 5 | 0.10 | 3.00 | | 0.01 | 0.99 | 1.00 | 16.18 | 85.48 | 9.21 |
| 6 | 0.10 | 3.00 | | 0.02 | 0.99 | 1.00 | 16.16 | 85.63 | 9.21 |
| 7 | 0.10 | 3.00 | | 0.3 | 0.99 | 1.00 | 16.15 | 85.76 | 9.21 |

What is claimed is:

1. A method for increasing attenuation in a fermented beverage comprising adding a transglucosidase to a maltose depleted fermentate.

2. The method of claim 1 wherein the fermented beverage is a beer.

3. The method of claim 2 wherein the fermentate has a concentration of maltose of less than 0.01% (w/w).

4. The method of claim 2 or 3 wherein less than 3 U/ml of transglucosidase is added to the fermentate.

5. The method of claim 4 wherein less than 1 U/ml of transglucosidase is added to the fermentate.

6. The method of claim 5 wherein less than 0.5 U/ml of transglucosidase is added to the fermentate.

7. The method of any of claims 2 to 6 wherein the transglucosidase is added after more than 50% of the total fermentation time.

8. The method of any of claims 2 to 7 wherein the beer has an RDF of at least 85%.

9. The method of claim 8 wherein the beer has an RDF of at least 86%.

10. The method of claim 9 wherein the beer has an RDF of at least 87%.

11. The method of claim 10 wherein the beer has an RDF of at least 88%.

12. The method of claim 11 wherein the beer has an RDF of at least 89%.

13. The method of any of claims 2 to 12 wherein the beer has an apparent extract of −0.20% or less by mass.

14. The method of any of claims 2 to 13 wherein the transglucosidase is added to the fermentate 4 days after initiation of fermentation.

15. The method of any of claims 1 to 14 further comprising adding a glucoamylase to the fermentate.

16. The method of claim 15 wherein the glucoamylase is added at the start of fermentation.

17. The method of any of claims 1 to 16 wherein the transglucosidase comprises a polypeptide having 70% or more sequence identity to SEQ ID NO:1 or a transglucosidase active fragment thereof.

18. The method of claim 17 wherein the polypeptide has 75% or more sequence identity to SEQ ID NO:1 or a transglucosidase active fragment thereof.

19. The method of claim 18 wherein the polypeptide has 80% or more sequence identity to SEQ ID NO:1 or a transglucosidase active fragment thereof.

20. The method of claim 19 wherein the polypeptide has 85% or more sequence identity to SEQ ID NO:1 or a transglucosidase active fragment thereof.

21. The method of claim 20 wherein the polypeptide has 90% or more sequence identity to SEQ ID NO:1 or a transglucosidase active fragment thereof.

22. The method of claim 21 wherein the polypeptide has 95% or more sequence identity to SEQ ID NO: 1 or a transglucosidase active fragment thereof.

23. The method of claim 22 wherein the polypeptide has 99% or more sequence identity to SEQ ID NO:1 or a transglucosidase active fragment thereof.

24. The method of claim 23 wherein the polypeptide has 100% sequence identity to SEQ ID NO: 1 or a transglucosidase active fragment thereof.
